(19) United States
(12) Patent Application Publication
(10) Pub. No.: US 2014/0323574 A1
(43) Pub. Date: Oct. 30, 2014

(54) INFANT FORMULA WITH HIGH SN-2 PALMITATE AND OLIGOFRUCTOSE

(71) Applicant: NESTEC S.A., Vevey (CH)

(72) Inventors: Manjiang Yao, Chester Springs, PA (US); Kalathur S. Ramanujam, Wayne, PA (US); Martin Jon Kullen, Pottstown, PA (US); Margaret Fitzgerald, Limerick (IE)

(21) Appl. No.: 14/357,625

(22) PCT Filed: Oct. 29, 2013
(86) PCT No.: PCT/IB2012/055975

§ 371 Date: May 12, 2014

(30) Foreign Application Priority Data
Nov. 10, 2011 (US) 61557950

(51) Int. Cl. A23L 1/29; A61K 31/20; A61K 31/702; A61K 38/38

(52) U.S. Cl. CPC A23L 1/296; A61K 38/38; A61K 31/20; A61K 31/702
USPC 514/558; 426/648

(57) ABSTRACT

An infant formula having a relatively high content of triglycerides having palmitic acid in the sn-2 position. The formula may include oligofructose. The formula may also include at least one omega 6 fatty acid and at least one omega 3 fatty acid. The formula may also have a relatively low protein content and an alpha-lactalbumin content similar to human milk The invention also includes a method for improving the stool consistency, increasing bifidobacteria in the colon, reducing potentially pathogenic bacteria in the colon and reducing calcium soaps in the stool of a formula-fed infant.

### BACKGROUND OF THE INVENTION

This invention relates to infant formula compositions having high sn-2 palmitate triglycerides and oligofructose.

Triglycerides are formed by ester bonds between glycerol, which has three hydroxyl groups, and three molecules of fatty acid. Triglycerides play an important role in metabolism as an energy source. In the intestine, triglycerides are split into monoacylglycerol and free fatty acids in a process called lipolysis, in which the free fatty acids are separated from the sn-1 and sn-3 positions of the triglyceride. Unsaturated free fatty acids are absorbed by the intestines much more easily than saturated fatty acids. When the sn-1 and/or sn-3 positions of the triglycerides include a high percentage of saturated fatty acid residues, the free fatty acids (such as palmitic acid) formed by lipolysis may combine with minerals, such as calcium or magnesium, to form soaps that make stools harder and more difficult to pass. This soap formation may also interfere with calcium absorption.

Human milk fat triglycerides contain approximately 20-25% palmitic acid residues, with about 70% present in the sn-2 position of the glyceride to which they are bonded. Vegetable oils are commonly used in infant formula instead of milk fat. Vegetable oil triglycerides typically have a high percentage, typically 80-85% or more, of palmitic acid present in the sn-1 or sn-3 position. Therefore, the free fatty acids formed during digestion of human milk are primarily unsaturated fatty acids, whereas the fatty acids released during digestion of vegetable oils are largely saturated fatty acids that may combine with calcium to form soaps. This may explain why infants that are breast fed are known to have softer stools than formula-fed infants.

Linoleic acid (LA), the predominant omega 6 fatty acid and alpha linolenic acid (ALA), the predominant omega 3 fatty acid, are essential for normal growth and development of human beings. Both LA and ALA fatty acids are metabolically distinct, cannot be synthesized in the human body and must be obtained from the diet of a mammal. During evolutional history, there was a general balance in the diet of a human with regard to the intake of LA and ALA and the ratio of LA to ALA was from about 1 to about 1. Due to modern agricultural methodologies and ease of transportation, the consumption of vegetable oils high in LA (corn, sunflower, safflower, soybean oils) increased, dramatically shifting the ratio of LA:ALA mammalian intake from about 1:1 to about 10-25 LA to about 1 ALA in many mammalian diets, particularly in the Western societies.

It is desirable to have an infant formula in which the triglycerides have more sn-2 palmitate and less sn-1 and sn-3 palmitate than is present in vegetable oil.

In infant formulations, it is desirable to optimize the fat blend using beta palmitate fat to improve calcium accretion and decrease stool soap fatty acids. Also, the inclusion of at least one omega 6 fatty acid and at least one omega 3 fatty acid in a ratio from about 6 to about 1 will provide short and long-term health benefits including improved docosahexaenoic acid (“DHA”) bioavailability.

A variety of vegetable-derived triglycerides having these characteristics are known in the art.

EP 1237419 describes an infant formula containing an easily digestible lipid component that does not contain high amounts of saturated fatty acids in the sn-1 and sn-3 position of glycerol, a viscosity improving component, a protein component containing less than 0.75 g of phosphorus per 100 g of protein, and/or a prebiotic component, such as an oligosaccharide.

WO 2005/036987 describes a new fat-based preparation comprising a mixture of vegetable-derived triglycerides having up to 38% palmitic acid residues with at least 60% of the palmitic acid residues in the sn-2 position of the glyceride, preferably with the sn-1 and sn-3 positions occupied by at least 70% unsaturated fatty acid residues, such as oleate.

WO 2006/114791 describes human milk fat substitutes for infant formula having vegetable-derived triglycerides with less than 50% of the fatty acid residues in the sn-2 position being saturated and/or the saturated fatty acid residues in the sn-2 position are less than 43.5% of the total saturated fatty acid residues.

Oligofructose is an oligosaccharide consisting of fructose units having a relatively low degree of polymerization. Oligofructose is well-known in the art and is commercially available.

U.S. Pat. No. 7,651,716 discloses an infant formula having 2.2-2.5 g/L alpha-lactalbumin (about 0.3-0.4 g/100 kcal), and that this is similar to human milk. A total protein content of 2.0-2.4 g/100 kcal of infant formula is also disclosed.

Methods for producing triglyceride compositions having a relatively high percentage of saturated fat residues, such as palmitic acid, in the sn-2 position are described in U.S. Pat. No. 5,658,768, WO2007/029015, WO2007/029018, WO2007/029020, and WO2008/104381. A commercially available composition sold by Lipid Nutrition is Betapol™ B-55, which is a triglyceride mixture derived from vegetable oil in which at least 54% of the palmitic acid is in the sn-2 position of the glycerol molecule.

### SUMMARY OF THE INVENTION

The present invention comprises an infant formula comprising, per 100 kcal of infant formula:
a) approximately from about 5 to about 6 g of fat, wherein at least 7.5 wt % of the total fat consists of triglycerides having palmitic acid in the sn-2 position;
b) optionally, at least about 0.4 g of oligofructose; and,
c) approximately from about 1.8 to about 2.2 g of total protein, optionally including approximately from about 0.3 to about 0.4 g of alpha-lactalbumin.

The present invention comprises an infant formula comprising, per 100 kcal of infant formula:
a) approximately 5-6 g of fat, wherein at least 7.5 wt % of the total fat consists of triglycerides having palmitic acid in the sn-2 position;
b) optionally, at least about 0.4 g of oligofructose;
c) approximately from about 1.8 to about 2.2 g of total protein, optionally including approximately from about 0.3 to about 0.4 g of alpha-lactalbumin; and,
d) at least one omega 6 fatty acid and at least one omega 3 fatty acid in a ratio of about 6 to about 1.

The present invention also comprises a method for improving the stool consistency of an infant comprising administering to said infant the infant formula of this invention.

The present invention further comprises a method for reducing the amount of calcium soaps in the stool of an infant comprising administering to said infant the infant formula of this invention.

The present invention additionally comprises a method for increasing the amount of beneficial bifidobacteria in the colon of an infant.

Other important aspects of the present invention will be apparent from the description below.

### BRIEF DESCRIPTION OF THE DRAWINGS

FIG. 1 is a bar graph showing the change in fecal bifidobacteria for infants fed various infant formulas and human milk.

FIG. 2 is a bar graph showing stool consistency for infants fed various infant formulas and human milk.

FIG. 3 is a bar graph showing the amount of palmitic acid soaps in the stool of infants fed various infant formulas and human milk.

### DETAILED DESCRIPTION OF THE INVENTION

The term “oligofructose” as used herein refers to a fructose oligomer having a degree of polymerization of from 2 to 10, for example a degree of polymerization of from 2 to 8.

The term “sn-2 palmitate” as used herein refers to palmitic acid in the sn-2 position of the triglyceride to which it is bonded.

The term “infant formula” as used herein refers to a nutritional formulation (either in the form of a liquid or in the form of a dry powder that may be reconstituted to form a liquid infant formula upon addition of water) that provides complete nutrition for an infant and is suitable to feed an infant, and which meets the US or EU standards for infant formula. Such formulae are well-known in the art.

Typically, an infant formula in a ready-to-consume liquid form provides 60-70 kcal/100 ml. Infant formula typically comprises, per 100 Kcal: about 1.8-4.5 g protein; about 3.3-6.0 g fat (lipids); about 300-1200 mg linoleic acid; about 9-14 g carbohydrates selected from the group consisting of lactose, sucrose, glucose, glucose syrup, starch, maltodextrins and maltose, and combinations thereof; and essential vitamins and minerals. Lactose may be the pre-dominant carbohydrate in an infant formula. For example, a liquid infant formula may contain about 67 kcal/100 ml. In some embodiments, infant formula may comprise about 1.8-3.3 g protein per 100 Kcal. Infant formula may be in the form of a powder which can be reconstituted into a ready-to-feed liquid by adding an amount of water that results in a liquid having about 67 kcal/100 ml.

An infant formula may also comprise nucleotides selected from cytidine 5′-monophosphate (CMP), uridine 5′-monophosphate (UMP), adenosine 5′-monophosphate (AMP), guanosine 5′-monophosphate (GMP) and inosine 5′-monophosphate (IMP), and mixtures thereof. Infant formula may also comprise lutein, zeaxanthin, fructo-oligosaccharides, galacto-oligosaccharides, sialyl-lactose, and/or fucosyl-lactose. Long chain polyunsaturated fatty acids, such as docosahexaenoic acid (DHA) and arachidonic acid (AA) may be included in infant formula. Infant formula may also include free amino acids. Infant formula may also include other ingredients well-known in the art.

In one embodiment, the infant formula of this invention comprises about 5-6 g per 100 kcal of fat (triglycerides), with at least about 7.5 wt % of this fat, for example about 7.5-12.0%, consisting of palmitic acid in the sn-2 position of a triglyceride. In some embodiments, about 7.8-11.8%, about 8.0-11.5 wt %, about 8.5-11.0% or about 9.0-10.0 wt % of the fat is palmitic acid in the sn-2 position of a triglyceride.

In some embodiments, palmitic acid comprises from about 15 to about 25%, such as from about 15 to about 20%, of the total fatty acids content of the formula, by weight, and at least from about 30%, for example, from about 35 to about 43% of the total palmitic acid content is in the sn-2 position.

In some embodiments, the infant formula further comprises at least one omega 6 fatty acid and at least one omega 3 fatty acid in a ratio of about 6 to about 1. In one embodiment, at least one omega 6 fatty acid comprises from about 10 to about 15% by weight of the total fatty acids and at least one omega 3 fatty acid comprises from about 1.2% to about 3.6% of the total fatty acids. In some embodiments, the infant formula comprises at least one omega 6 fatty acid present from about 2 to about 4% of the total weight and at least one omega 3 fatty acid present from about 0.3% to about 0.6% of the total weight.

The fat in the infant formula of this invention comprises a variety of triglycerides typically found in milk and/or infant formula. The most common fatty acid residues in the triglycerides are palmitic and oleic acids. Fatty acid residues in addition to oleic and palmitic acids that are present include, but are not limited to linoleic acid, alpha linolenic acid, lauric acid, myristic acid, docosahexaenoic acid, and arachidonic acid.

A commercially available composition sold by Lipid Nutrition is Betapol™ B-55, which is a triglyceride mixture derived from vegetable oil in which at least 54% of the palmitic acid is in the sn-2 position of the glycerol molecule. In one embodiment, the fat content of the formula of this invention is about 40-50% Betapol™ B-55 by weight, for example from about 43% to about 45% by weight. Those skilled in the art will appreciate that the percentage of the high sn-2 fat used and the total amount of sn-2 palmitate in the formula may vary, and that a different high sn-2 palmitate oil may be used, without departing from the spirit and scope of the invention.

Although feeding an infant a formula containing a high percentage of sn-2 palmitate helps to produce softer stools and growth of bifidobacteria in the colon, the combination of high sn-2 palmitate with oligofructose provides significantly superior stool softening and growth of bifidobacteria in the colon of formula-fed infants. A significant reduction in the amount of potentially pathogenic bacteria can also be achieved. It has been discovered that feeding an infant a high sn-2 palmitate-containing infant formula containing from about 3 to about 5 g/L, or from about 0.4 to about 0.7 g/100 kcal, of oligofructose is more beneficial than feeding the infant the same formula without oligofructose.

The present invention results in a reduction of fecal palmitic acid soaps, which may lead to reduced constipation and improved gastrointestinal tolerance, compared to standard infant formula.

The infant formula of this invention contains at least about 0.4 g of oligofructose of oligofructose per 100 kcal. In some embodiments, it contains from about 0.4 to about 0.9 g, from about 0.4 to about 0.7 g, from about 0.4 to about 0.5 g, from about 0.7 to about 0.8 g, or from about 0.7 to about 0.9 g, oligofructose per 100 kcal. The oligofructose has a degree of polymerization of from 2 to 10. In one embodiment, at least 90% of the oligofructose has a degree of polymerization of from 2 to 8.

Recent infant clinical studies have shown that nutritional formulas containing at least one omega 6 fatty acid and at least one omega 3 fatty acid in a ratio of from about 6 to about 1 increased DHA accretion in erythrocytes and plasma. A balanced ratio of about 6:1 of omega 6 fatty acid to omega 3 fatty acid may also provide long term health benefits including protection against cardiovascular disease. Such balance will be achieved by formulating the present invention with vegetable oil fat sources that have omega 6 fatty acid content, such as, for example, soybean oil and sunflower oil, and omega 3 fatty acid content, for example, rapeseed, canola, flaxseed, chia, perlla or walnuts. A unique fat blend with 5 different oils will be used to achieve the modified fat blend

In one embodiment, the infant formula of this invention comprises from about 1.8 to about 2.2 g of total protein per 100 kcal, for example, about from 1.8 to about 2.1 g or from about 1.9 to about 2.1 g protein per 100 kcal, wherein from about 0.3 to about0.4 g/100 kcal of protein is alpha-lactalbumin. The infant formula of this invention may be in the form of a ready-to-feed liquid, or may be a liquid concentrate or powdered formula that can be reconstituted into a ready-to-feed liquid by adding an amount of water that results in a liquid having about 67 kcal/100 ml. The infant formula of this invention includes all the ingredients that are required by law in the US or EU, including but not limited to certain vitamins, minerals, and essential amino acids. It may also include nucleotides, such as CMP, UMP, AMP, GMP and IMP, lutein, zeaxanthin, and other ingredients known in the art.

The following examples are presented to illustrate certain embodiments and features of the present invention, but should not be construed as limiting the scope of this invention.

### Example 1

1. Control Formula

As a ready-to-feed liquid infant formula, the Control Formula has 670 kcal/L. Ingredients are shown below:

| Ingredients | Per 100 Kcal | Per Liter |
|---|---|---|
| Total Protein | 2.0 g | 13.4 g |
| (alpha-lactalbumin) | (0.3 g) | (2.3 g) |
| Total Fat | 5.4 g | 36 g |
| (sn-2 palmitate) | (0.19 g) | (1.3 g) |
| Total Carbohydrates | 11 g | 73 g |

The Control Formula also includes essential amino acids, minerals and trace elements, nucleotides, and various optional ingredients and food additives commonly used in infant formula.

2. High sn-2 Formula

This formula is the same as the Control Formula, except that 9.6 wt % of the fat is sn-2 palmitate. This is accomplished by using fat that is 57% vegetable oil and 43% Betapol™ B-55 in which about 55% of the palmitic acid is in the sn-2 position.

3. High sn-2+ Oligofructose Formula A

This formula is the same as the High sn-2 Formula except that it includes 3.0 g/L (0.4 g per 100 kcal) oligofructose.

4. High sn-2+ Oligofructose Formula B

This formula is the same as the High sn-2 Formula except that it includes 5.0 g/L (0.7 g per 100 kcal) oligofructose.

5. High sn-2+ Oligofructose+Omega 6 and Omega 3 Formula C

This formula is the same as the High sn-2 Formula except that Formula C includes from about 3 to about 5 g/L (about 0.75 g per 100 kcal) oligofructose and about 4.5 g/reconstituted liter(RL) (about 0.65 g per 100 kcal) omega 6 fatty acid and about 730 mg/RL (110 mg per 100 kcal) omega 3 fatty acid.

### Example 2

A study is run to compare three infant formulas: High sn-2 Formula, High sn-2 30 Oligofructose Formula A, and High sn-2+ Oligofructose Formula B to the Control Formula, in a double-blind randomized controlled design. Formula C was not tested. Human breast milk was also included as a non-randomized reference group. Three hundred formula-fed infants are randomly assigned to four groups (75 per group), and each group is fed a different infant formula of Example 1 (Control Formula, High sn-2 Formula, High sn-2+ Oligofructose Formula A, or High sn-2+ Oligofructose Formula B) for eight weeks. Another group of seventy five infants is fed human breast milk for 8 weeks. All the infants are healthy term infants 7-14 days old at the start of the study.

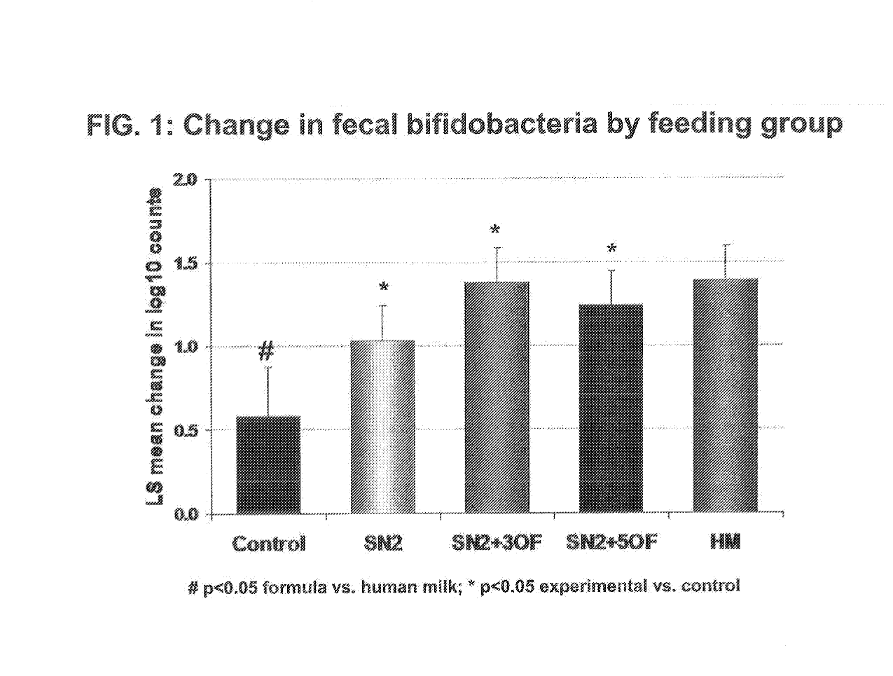

As one of the outcome measures, in a subgroup of 170 infants, stool samples were collected at the baseline and week-8 visits and analyzed for fecal microflora using the fluorescent in situ hybridization (FISH) method. It is well established that FISH is a highly valuable tool for the specific and rapid detection of microorganisms in clinical samples without cultivation. Analysis of fecal microflora in the subgroup of infants showed that infants fed high sn-2 palmitate formula, both with (“SN2+OF”) or without (“SN2”) the added oligofructose, had a significantly greater increase in fecal bifidobacteria concentrations over 8-week period compared to the control group (p=0.033 for SN2 group; p≦0.002 for SN2+OF groups), and did not differ significantly from that of human milk (“HM”)-fed infants, as shown in FIG. 1. This finding indicates that the high sn-2 palmitate formula alone had a stimulatory effect on the growth of beneficial bifidobacteria, resulting in increased amounts of bifidobacteria in the digestive system, more closely resembling the natural amounts in the colon of a breastfed infant.

To measure stool biochemical composition infant stool samples were collected during Week 8 and analyzed for the following stool composition variables: individual soap fatty acids; non-soap fatty acids; total fatty acids; soap lipids; non-soap lipids; minerals: calcium, magnesium, and phosphorus; nitrogen; stool solids; and stool moisture. The two most important stool composition evaluations were palmitic acid soaps and total fatty acid soaps (predetermined in the Statistical Analysis Plan). Utilizing a kit provided to the parents/legal guardians, stool sample was collected at home from all study infants during the 5-day period prior to the final visit at Week 8. Infants were fitted with diapers containing a strip of Tegaderm tape in the area where the infant has bowel movements to help the stool to stay in the diaper. Parents scooped freshly passed stool during the collection period, deposited the samples in amber plastic bags, weighed each bag on the portable scale, and stored the bag in freezer section of the home refrigerator.

Once at least 30 g of stool had been collected, study personnel transferred the frozen samples in the collection-kit cooler bag to the study clinic and stored in a −20° C. freezer. Samples were shipped frozen on dry-ice to Covance Laboratories, Inc., Madison, Wis., USA where samples were analyzed using methods cited by Quinlan et al. to determine total soap fatty acids; non-soap fatty acids; total fatty acids; soap lipids; and non-soap lipids. Standard analytical procedures were used for the determination of stool moisture and solids. Mineral contents including calcium, phosphorous, and magnesium were determined by ICP Emission Spectrometry following AOAC International (AOAC) Official Methods of Analysis. Nitrogen was determined by the Dumas Method utilizing a combustion-detection technique.

For determining soap fatty acids and non-soap fatty acids, stool samples were thawed, homogenized by stirring, and the weight of the wet stool was recorded. The sample was then lyophilized and the dry-stool weight was recorded. A 0.5- to 1.0-g freeze-dried sample of stool was transferred to an extraction thimble and extracted by solvent reflux to obtain neutral lipids, including non-soap free fatty acids. The sample remaining in the thimble was treated with acetic acid to release the soap fatty acids, which were then isolated by a second solvent reflux step. Internal standards were added to the two extracts and the free acids were absorbed onto an anhydrous alkaline exchange resin. The free acids were then extracted from the resin and converted to methyl esters using hydrochloric acid and methanol. The resulting fatty acid methyl esters were analyzed by gas chromatography. The fatty acids of interest were: lauric (C12:0), myristic (C14:0), palmitic (C16:0), stearic (C18:0), oleic (C18:1), and linoleic (C18:2). The principal fatty acid of interest was C16:0; therefore for the assay, the limit of quantitation (LOQ) was defined as the sample concentration corresponding to the lowest C16:0 calibration standard of 0.05%, which was then adjusted for the sample weight. The acceptance criteria with respect to recovery and coefficients of variability (CVs) for fatty acids were +/−30% for the performance of replicate analyses related to accuracy as compared to previously established value of targets in quality control (QC) samples. Repeatability relative standard deviation (RSD) values for dry quality control (QC) level of C16:0 ranged from 2.9% to 12.6%. Total soap fatty acids were calculated as the sum of all measured individual soap fatty acids. Total non-soap fatty acids were calculated as the sum of all measured individual non-soap fatty acids. Total fatty acids were calculated as total non-soap fatty acids plus total soap fatty acids. Results were expressed as mg per g of dry stool weight.

To measure stool characteristics (consistency and frequency) parents/legal guardians completed a 3-day stool diary on three consecutive days immediately prior to study visits at Week 4 and Week 8 in which they recorded the number of stools per day and the consistency of stools as 1=watery, 2=runny, 3=mushy soft, 4=formed, or 5=hard based on standardized pictures of representative stools that accompanied the instructions for the diary. The five stool consistency options were based on a validated 5-point scale of Weaver et al..

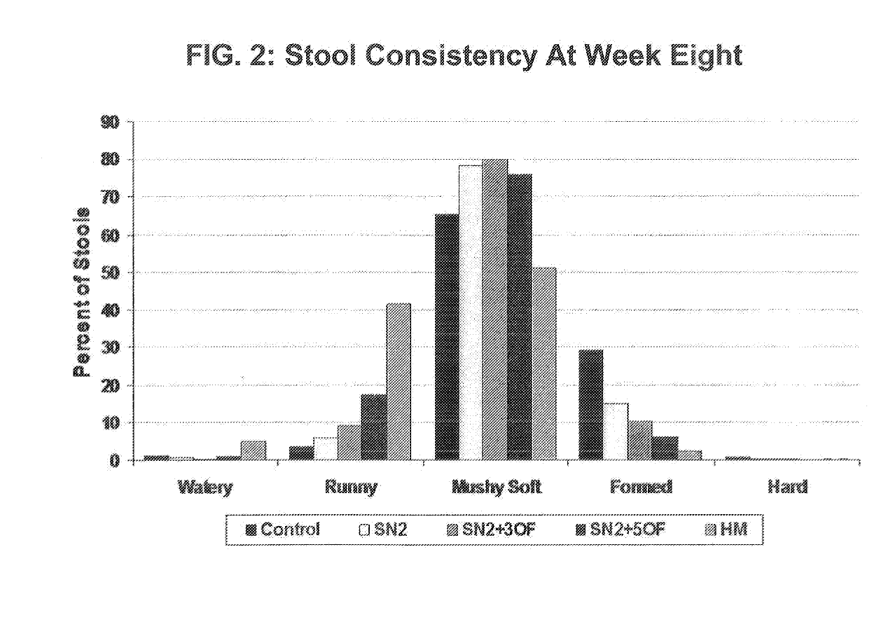

As shown in FIG. 2, infants fed a high sn-2 palmitate formula without oligofructose had significantly less formed stools than the control group, and the formulas containing both high sn-2 palmitate and oligofructose produced even less formed stools, approaching the level for the human milk group.

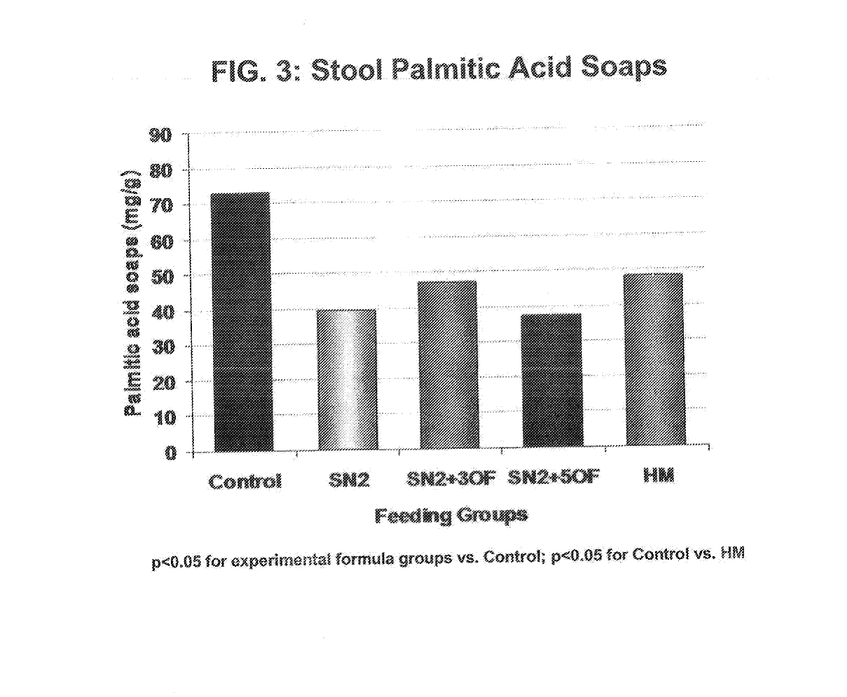

All the high sn-2 palmitate formulas, both with and without oligofructose, produced significantly lower levels of palmitic acid soap than the control formula, comparable to human milk, as shown in FIG. 3.

Many variations of the present invention not illustrated herein will occur to those skilled in the art. The present invention is not limited to the embodiments illustrated and described herein, but encompasses all the subject matter within the scope of the appended claims.

1. An infant formula comprising, per 100 kcal of infant formula, from about 5 to about 6 g of fat, and about 7.5 wt % to about 12.0 wt % of total fat consists of palmitic acid in the sn-2 position.

2. An infant formula comprising, per 100 kcal of infant formula:
from about 5 to about 6 g of fat, wherein at least about 7.5 wt % of total fat consists of palmitic acid in the sn-2 position;
at least about 0.4 g of oligofructose; and
from about 1.8 to about 2.2 g of total protein.

3. The infant formula of claim 2 wherein the protein comprises from about 0.3 g to about 0.4 g of alpha-lactalbumin per 100 kcal.

4. The infant formula of claim 3 wherein the total protein is about 1.9 to about 2.1 g per 100 kcal.

5. The infant formula of claim 2 wherein from about 7.5 wt % to about 12.0 wt % of the total fat comprises palmitic acid in the sn-2 position.

6. The infant formula of claim 5 wherein the infant formula comprises from about 0.4 to about 0.7 g of oligofructose per 100 kcal.

7. The infant formula of claim 1 wherein from about 7.8 wt % to about 11.8 wt % of the total fat comprises palmitic acid in the sn-2 position.

8. The infant formula of claim 2 wherein at least about 30% of the palmitic acid in the fat is in the sn-2 position.

9. The infant formula of claim 2 wherein:
the total protein is of from about 1.9 to about 2.1 g and comprises about 0.3 g alpha-lactalbumin, per 100 kcal;
from about 7.5% to about 12.0% by weight of the total fat consists of palmitic acid in the sn-2 position;
at least about 30% of the palmitic acid in the fat is in the sn-2 position; and
the formula comprises from about 0.4 to about 0.5 g oligofructose per 100 kcal.

10. The infant formula of claim 2 wherein:
the total protein is of from about 1.9 to about 2.1 g and comprises about 0.3 g alpha-lactalbumin, per 100 kcal;
from about 7.5% to about 12.0% by weight of the total fat consists of palmitic acid in the sn-2 position;
at least about 30% of the palmitic acid in the fat is in the sn-2 position; and
the formula comprises from about 0.7 to about 0.8 g oligofructose per 100 kcal.

11. The infant formula of claim 2 wherein about 35 to about 43% of the palmitic acid in the fat is in the sn-2 position.

12. The infant formula of claim 2 wherein at least about 90% of the oligofructose has a degree of polymerization of from 2 to 8.

13. A method for improving the stool consistency of an infant comprising administering to the infant an infant formula comprising, per 100 kcal of infant formula, from about 5 to about 6 g of fat, and about 7.5 wt % to about 12.0 wt % of total fat consists of palmitic acid in the sn-2 position.

14. A method for reducing the amount of calcium soaps in the stool of an infant comprising administering to the infant an infant formula comprising, per 100 kcal of infant formula, from about 5 to about 6 g of fat, and about 7.5 wt % to about 12.0 wt % of total fat consists of palmitic acid in the sn-2 position.

15. A method of increasing the fecal bifidobacteria concentration in an infant's stool comprising administering to the infant an infant formula comprising, per 100 kcal of infant formula, from about 5 to about 6 g of fat, and about 7.5 wt % to about 12.0 wt % of total fat consists of palmitic acid in the sn-2 position.

16. A method of reducing the amount of potentially pathogenic bacteria in an infant's colon comprising administering to the infant an infant formula comprising, per 100 kcal of infant formula, from about 5 to about 6 g of fat, and about 7.5 wt % to about 12.0 wt % of total fat consists of palmitic acid in the sn-2 position.

17. An infant formula comprising, per 100 kcal of infant formula:
from about 5 to about 6 g of fat, wherein at least about 7.5 wt % of total fat consists of palmitic acid in the sn-2 position;
at least about 0.4 g of oligofructose;
from about 1.8 to about 2.2 g of total protein; and
at least one omega 6 fatty acid and at least one omega 3 fatty acid in a ratio of about 6 to about 1.

18. An infant formula comprising, per 100 kcal of infant formula:
approximately from about 5 to about 6 g of fat, wherein at least about 7.5 wt % of the total fat consists of triglycerides having palmitic acid in the sn-2 position;
from about 1.8 to about 2.2 g of total protein; and
at least one omega 6 fatty acid and at least one omega 3 fatty acid in a ratio of about 6 to about 1.
